(19) United States
(12) Patent Application Publication
(10) Pub. No.: US 2007/0117171 A1
(43) Pub. Date: May 24, 2007

(54) METHOD OF MANUFACTURING A DIAGNOSTIC TEST STRIP

(76) Inventors: Greta Wegner, St. Anthony, MN (US); Natasha Popovich, Pompano Beach, FL (US); Dennis Slomski, Wellington, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/530,542

(22) Filed: Sep. 11, 2006

Related U.S. Application Data
(60) Provisional application No. 60/716,120, filed on Sep. 13, 2005.

(51) Int. Cl. C12Q 1/54; B05D 3/00

(52) U.S. Cl. 435/14; 427/2.11

(57) ABSTRACT

A method for manufacturing a test strip is provided. The method comprises selecting a test strip substrate material and positioning the test strip substrate material a predetermined distance from a matrix material disposed on a second substrate, wherein at least one enzyme to be deposited on the test strip substrate and having glucose as an enzymatic substrate is held within the matrix material. A laser pulse is directed towards the matrix material to release at least a portion of the at least one enzyme having glucose as an enzymatic substrate from the matrix material and deposit the enzyme on the test strip substrate.

### DESCRIPTION

This application claims priority to U.S. Provisional Patent Application No. 60/716,120, filed on Sept. 13, 2005.

### TECHNICAL FIELD

The present invention relates to the field of diagnostic testing and, more particularly, to diagnostic testing systems using electronic meters.

### BACKGROUND

Electronic testing systems are commonly used to measure or identify one or more analytes in a sample. Such testing systems can be used to evaluate medical samples for diagnostic purposes and to test various non-medical samples. For example, medical diagnostic meters can provide information regarding the presence, amount, or concentration of various analytes in human or animal body fluids. In addition, diagnostic test meters can be used to monitor analytes or chemical parameters in non-medical samples such as water, soil, sewage, sand, air, or any other suitable sample.

Diagnostic testing systems typically include both a test media, such as a diagnostic test strip, and a test meter configured for use with the test media. Suitable test media may include a combination of electrical, chemical, and/or optical components configured to provide a response indicative of the presence or concentration of an analyte to be measured. For example, some glucose test strips include electrochemical components, such as glucose specific enzymes, buffers, and one or more electrodes. The glucose specific enzymes may react with glucose in a sample, thereby producing an electrical signal that can be measured with the one or more electrodes. The test meter can then convert the electrical signal into a glucose test result.

There is a demand for improved test media. For example, in the blood glucose testing market, consumers consistently insist on test media that require smaller sample sizes, thereby minimizing the amount of blood needed for frequent testing and further preventing erroneous tests due to inadequate sample size. In addition, in all diagnostic testing markets, consumers prefer faster, cheaper, more durable, and more reliable testing systems.

Current methods of manufacturing diagnostic test media may have inherent limits. For example, current methods for producing test media electrodes and depositing enzymes or other chemicals may have limited spatial resolution and production speeds. Furthermore, some production processes cannot be used to deposit some enzymes, chemicals, and electrodes. In addition, some production processes may be used to produce or deposit some test media components, such as electrodes or enzymes, while being incompatible with other components. Therefore, some test media production processes may require multiple production techniques, thereby increasing production cost and time, and decreasing product throughput.

Accordingly, there is a need for improved methods of manufacturing diagnostic testing systems.

### SUMMARY

A first aspect of the present invention includes a method for manufacturing a test strip. The method comprises selecting a test strip substrate material and positioning the test strip substrate material a predetermined distance from a matrix material disposed on a second substrate, wherein at least one enzyme to be deposited on the test strip substrate and having glucose as an enzymatic substrate is held within the matrix material. A laser pulse is directed towards the matrix material to release at least a portion of the at least one enzyme having glucose as an enzymatic substrate from the matrix material and deposit the enzyme on the test strip substrate.

A second aspect of the present invention includes a method for manufacturing a test strip. The method comprises selecting a test strip substrate material and positioning the test strip substrate material a predetermined distance from a matrix material disposed on a second substrate, wherein at least one electrode material to be deposited on the test strip substrate is held within the matrix material. A laser pulse is directed towards the matrix material to release at least a portion of the at least one electrode material released from the matrix material and deposit the electrode material on the test strip substrate.

A third aspect of the present invention includes a method for manufacturing a test strip. The method comprises selecting a test strip substrate material, positioning the test strip substrate material a predetermined distance from an electrode matrix material disposed on a second substrate, wherein at least one electrode material to be deposited on the test strip substrate is held within the electrode matrix material. A laser pulse is directed towards the electrode matrix material to release at least a portion of the at least one electrode material from the electrode matrix material and deposit the electrode material on the test strip substrate. The method further comprises positioning the test strip substrate material a predetermined distance from an enzyme matrix material disposed on a third substrate, wherein at least one enzyme to be deposited on the test strip substrate and having glucose as an enzymatic substrate is held within the enzyme matrix material. A laser pulse is directed towards the enzyme matrix material to release at least a portion of the at least one enzyme having glucose as an enzymatic substrate from the enzyme matrix material and deposit the enzyme on the test strip substrate.

A fourth aspect of the present invention includes a method for manufacturing a test strip. The method comprises selecting a test strip substrate material and applying an aerosolized material including at least one enzyme having glucose as an enzymatic substrate to the test strip substrate.

A fifth aspect of the present invention includes a method for manufacturing a test strip. The method comprises selecting a test strip substrate material and applying an aerosolized material including at least one electrode material to the test strip substrate.

A sixth aspect of the present invention includes a method for manufacturing a test strip. The method comprises selecting a test strip substrate material, applying an aerosolized material including at least one electrode material to the test strip substrate, and applying an aerosolized material including at least one enzyme having glucose as an enzymatic substrate to the test strip substrate.

Additional aspects and advantages of the invention will be set forth in part in the description which follows, and in part will be apparent from the description, or can be learned by practice of the invention. The advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A illustrates test media that can be produced using the methods of the present disclosure.

FIG. 1B illustrates a test meter that can be used with test media produced according to the methods of the present disclosure.

FIG. 1C illustrates a test meter that can be used with test media produced according to the methods of the present disclosure.

FIG. 2 illustrates another test media produced using the methods of the present disclosure.

FIG. 3 illustrates a direct-write laser deposition system, according to an exemplary disclosed embodiment.

FIG. 4 illustrates a pulsed laser deposition system, according to an exemplary disclosed embodiment.

FIG. 5 illustrates a laser deposition process including a mask, according to an exemplary disclosed embodiment.

FIG. 6. illustrates an aerosol deposition system, according to an exemplary disclosed embodiment.

FIG. 7 illustrates an aerosol deposition process including a mask, according to an exemplary disclosed embodiment.

FIG. 8 provides a photograph of a sample test media including an electrode produced using the methods of the present disclosure.

FIG. 9 provides a photograph of a sample test media including an electrode produced using the methods of the present disclosure.

FIG. 10 provides a photograph of an enzyme formulation deposited using the methods of the present disclosure.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

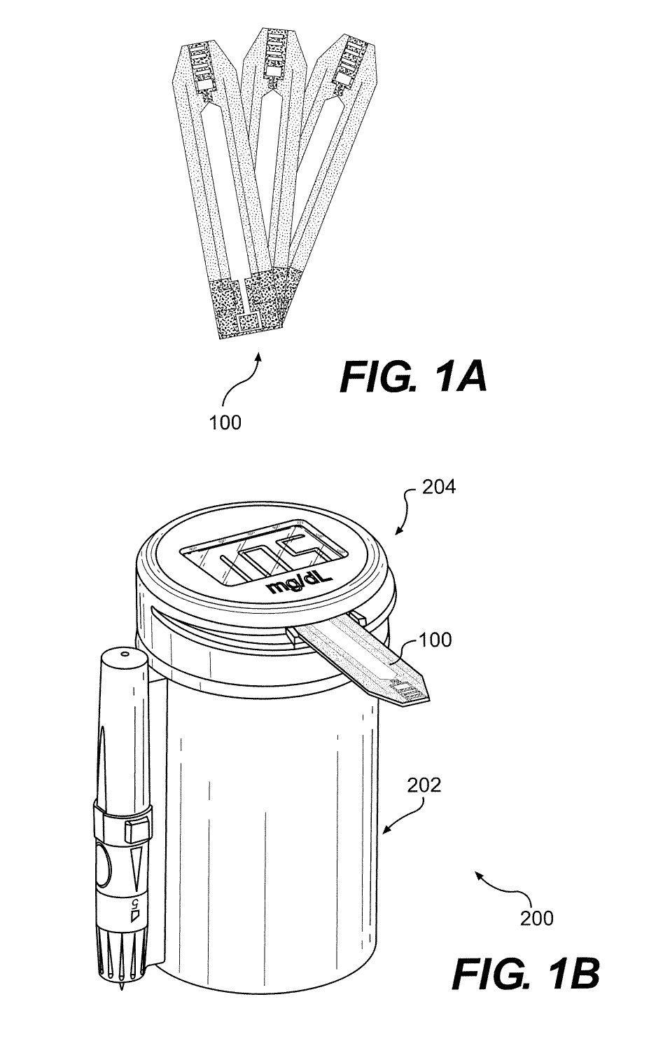

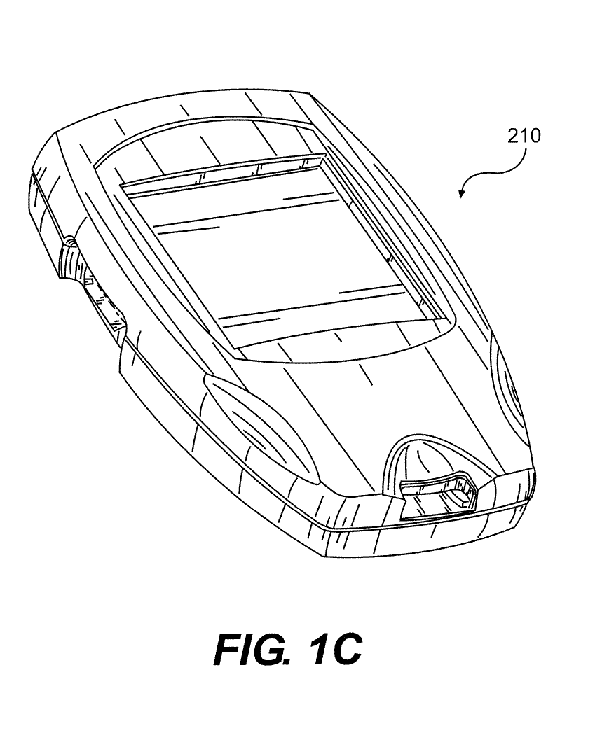

The present disclosure provides a method for producing diagnostic test media 100, as shown in FIG. 1A. The test media 100 of the present disclosure may be used with a suitable test meter 200, 210, as shown in FIGS. 1B and 1C, to detect or measure the concentration of one or more analytes. The one or more analytes may include a variety of different substances, which may be found in biological samples, such as blood, urine, tear drops, semen, feces, gastric fluid, sweat, cerebrospinal fluid, saliva, vaginal fluids (including suspected amniotic fluid), culture media, and/or any other biologic sample. The one or more analytes may also include substances found in environmental samples such as soil, food products, ground water, pool water, and/or any other suitable sample.

As shown, the test media 100 are test strips. However, the test media 100 may be provided in any suitable form including, for example, ribbons, tabs, discs, or any other suitable form. Furthermore, the test media 100 can be configured for use with a variety of suitable testing modalities, including electrochemical tests, photochemical tests, electrochemilluminescent tests, and/or any other suitable testing modality.

The test meter 200, 210 may be selected from a variety of suitable test media types. For example, as shown in FIG. 1B, the test meter 200 includes a vial 202 configured to store the test media 100. The operative components of the meter 200 may be contained in a meter cap 204. The meter cap 204 will contain the electrical meter components, can be packaged with the test meter 200, and can be configured to close and/or seal the vial 202. Alternatively, the test meter 210 can include a single unit that is separated from the test media storage vial, as shown in FIG. 1C. Any suitable test meter may be selected to provide a diagnostic test using the test media 100 produced according to the disclosed methods.

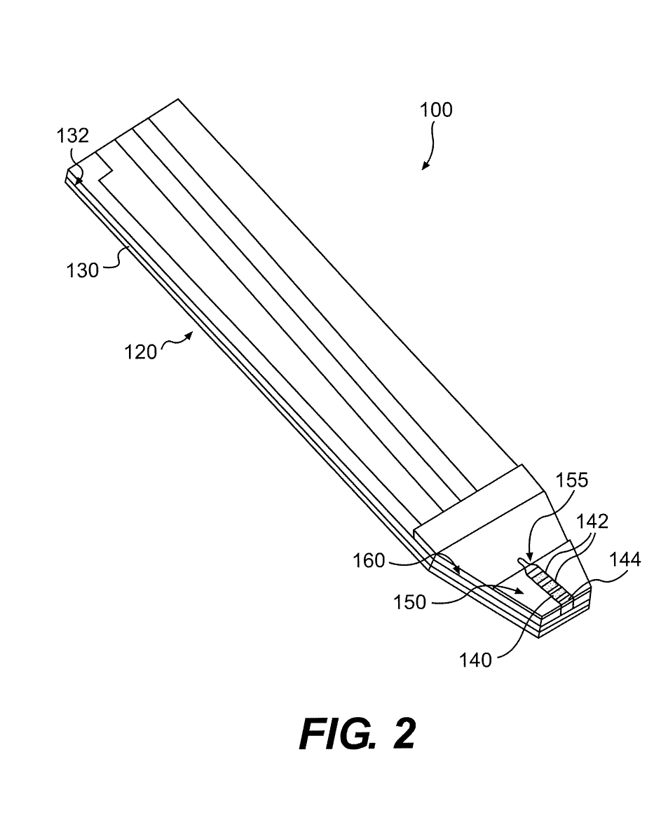

The test media 100 may include a number of components, as shown in FIG. 2. For example, the test media 100 may include a substrate material 120, which can be formed from one or more material layers 130, 132. The one or more material layers 130, 132 will provide desired physical, chemical, electrical, and/or thermal properties to the substrate material 120. Further, materials selected to produce the one or more layers 130, 132 of the substrate material 120 may be selected from a number of suitable material types, including a variety of different polymers, metals, and/or composite materials. In addition, the one or more layers 130, 132 may be produced from multiple different materials or may include a single material.

In some embodiments, the materials selected to produce the one or more layers 130, 132 will provide desired mechanical properties to the substrate material 120. For example, the one or more layers 130, 132 may include a material having a certain strength, rigidity, and/or fracture toughness to provide structural support to the substrate material 120. In other embodiments, the one or more layers 130, 132 may include a material having certain electrical or thermal properties. For example, the one or more layers 130, 132 may include a material having a certain electrical conductivity or resistivity.

In other embodiments, the one or more layers 130, 132 may include materials having certain chemical or biologic properties. For example, the one or more layers 130, 132 may include materials that are substantially inert, thereby limiting the effect of the materials on chemical or enzymatic reactions that may occur on the test media 100. Alternatively, the one or more layers 130, 132 may include materials that may catalyze or slow a reaction that may occur on the test media 100.

The test media 100 may further include one or more electrodes 140, 142, 144. The one or more electrodes 140, 142, 144 may be configured to facilitate measurement of electrical signals, i. e. currents or voltages, produced by an electrochemical reaction within a test strip reaction site 150. The one or more electrodes 140, 142, 144 can include a number of electrode types and configurations. For example, the one or more electrodes 140, 142, 144 can include a cathode 140 and an anode 144, as well as one or more additional electrodes 142. The specific number and type of electrodes may be selected based on the desired testing modality, the specific test meter type being used, and any other suitable parameter. Furthermore, the one or more electrodes 140, 142, 144 can include a number of shapes and configurations. For example, the one or more electrodes can include planar electrodes, interdigitated electrodes, or any other suitable electrode pattern.

The electrodes 140, 142, 144 can be produced from a number of suitable materials. For example, the electrode materials can include a variety of suitable conductive or semiconductive materials, including gold, silver, platinum, copper, palladium, doped silicon, carbon, carbon nanotubes, conductive polymers, and/or any other suitable electrode material. Further, suitable materials may be provided in a variety of different states. For example, suitable metals may be provided as solids, powders, and/or nanoparticles. The specific electrode materials may be selected based on cost, availability, compatibility with desired production processes, and/or compatibility with a desired testing modality.

To produce a signal indicative of the presence or concentration of an analyte in a sample to be tested, the test strip reaction site 150 may further include one or more substances configured to react with one or more analytes. For example, the reaction test site 150 may include one or more enzymes configured to react with an analyte such as glucose. Furthermore, the reaction test site 150 may include other additives, including salts, buffers, enzyme stabilizers, electrochemical mediators, color indicators, and/or any other chemical needed to facilitate production of a suitable test reaction.

The reaction test site 150 may have a shape and size configured to hold certain substances needed to react with an analyte to be tested. For example, the reaction test site 150 may include a well configured to secure a certain sample volume. In addition, the reaction test site 150 may include various configurations that can facilitate sample acquisition, proper sample placement, or needed fluid flow. For example, some test strips 100 may include one or more vent holes 155 configured to facilitate sample acquisition, capillary channels, or any other suitable configurations which may facilitate flow of some samples into a sample well. Any suitable test site shape and size may be used. In one embodiment, the test media 100 can include a substrate 120 having multiple layers 130, 132. Further, the reaction test site may 150 may be produced as a well formed as a cut out in a spacer layer 160 disposed on the substrate 120 (as shown in FIG. 2). Alternatively, the reaction test site 150 can include a well produced in one or more substrate layers 130, 132, and materials deposited in the reaction test site 150 using the methods of the present disclosure may be deposited in the reaction site well.

The test media 100 may be produced using a number of suitable manufacturing techniques. In one embodiment, a suitable test media substrate material 120 may be selected first. As noted above, the test media substrate material 150 may include one or more layers produced from a variety of suitable materials and having a variety of shapes, sizes, and configurations. Any suitable substrate material may be selected.

Next, one or more test media components may be produced on or applied to a selected region of the test media substrate material 120. The one or more test media components may include one or more test electrodes, reference electrodes, chemicals, enzymes, and/or any other component selected to facilitate measurement or detection of one or more selected analytes.

The test media components, including electrodes 140, 142, 144, chemicals, and/or enzymes, may be applied to or produced on the substrate material 120 sequentially or simultaneously. For example, in one embodiment, one or more electrodes 140, 142, 144 may be produced on a suitable test media substrate material 120 before applying suitable enzymes or chemicals to the substrate material 120. In some cases, production of electrodes 140, 142, 144 before deposition of enzymes may prevent damage to or inactivation of certain enzymes due to some electrode production processes. For example, some electrode production processes may include heat treatment, sintering, and/or laser trimming steps, which can inactivate or damage certain enzymes or chemicals.

In some embodiments, one or more test media components may be produced using a laser-assisted deposition technique. Laser-assisted deposition techniques can allow controlled deposition of electrode materials, enzymes, and/or other chemicals. Further, laser deposition may be used to produce component features having a certain spatial resolution, which may facilitate production of suitable component dimensions needed for small sample size or short reaction times. In addition, laser deposition can be used to deposit enzymes on suitable test media substrates 120 without inactivating or substantially damaging these enzymes.

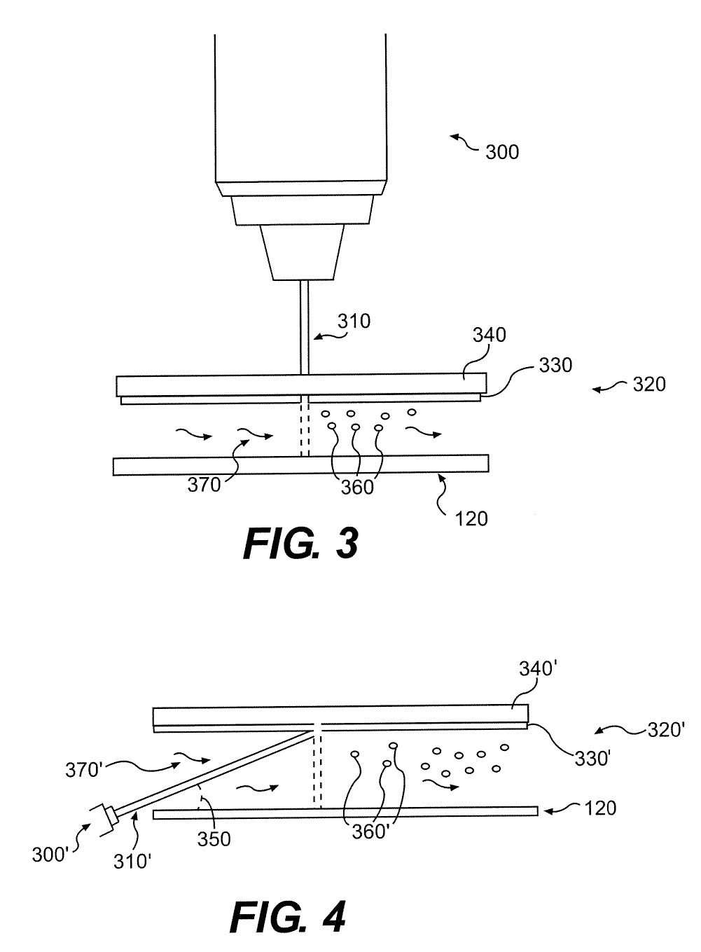

One suitable laser deposition technique is illustrated in FIG. 3. This deposition technique may be described as a laser direct-write process or laser forward transfer. Laser direct-write processes have been described in several U.S. patents, including U.S. Pat. No. 6,177,151 to Chrisey et al., U.S. Pat. No. 6,805,918 to Auyeung et al., and U.S. Pat. No. 6,905,738 to Ringeisen et al, each of which is herein incorporated by reference in its entirety.

In the laser direct write process of FIG. 3, a laser 300 will produce a laser pulse 310, which is directed through a target material 320. The laser pulse will release substances contained within the target material 320, thereby directing the selected substances contained within the target material 320 onto a suitable substrate 120. A variety of deposition parameters may be controlled to facilitate deposition of desired substances on the substrate 120.

Another suitable laser deposition technique is illustrated in FIG. 4. In this deposition technique, a laser pulse 310′ is directed at the target material 320′ at a certain angle 350, rather than through the target material 320′. This type of laser deposition process may be referred to as pulsed-laser deposition. Pulsed-laser deposition of glucose oxidase is described in Phadke et al., “Laser-assisted deposition of preformed mesoscopic systems, ” Materials Science and Engineering, C5: 237-241 (1998), herein incorporated by reference in its entirety. As in the laser direct-write technique of FIG. 4, the laser pulse 310′ will release substances contained within the target material 320′, thereby directing selected substances contained within the target material 320′ onto a substrate 120. Variations of both pulsed laser deposition and laser direct-write processes for biomaterials are described in Wu et al., “Laser transfer of biomaterials: Matrix-assisted pulsed laser evaporation (MAPLE) and MAPLE direct write,” Review of Scientific Instruments, 74(4): 2546-2557(2003), which is herein incorporated by reference in its entirety.

For both pulsed-laser deposition and laser direct-write processes, the substrate 120 can be provided in a number of suitable forms. For example, the substrate 120 may include a single sheet of substrate material which may be cut or otherwise divided into multiple test media 100. Alternatively, the substrate 120 may be sized for production of a single test media 100. Further, the substrate 120 may include multiple test media substrates placed side by side and optionally disposed on a sample holder, such at a tray, conveyor belt, or any other suitable holder.

In both laser-direct write and pulsed laser deposition, the substrate 120 may be positioned at a certain distance from the target material 320, 320′. The specific distance may be selected based on the desired size and configuration of the component feature to be produced. For example, in some embodiments, the substrate 120 may be positioned close to the target material 320, 320′ to produce a small component dimension. Alternatively, the substrate 120 may be positioned further from the target material to facilitate dispersion of the deposited substance or to produce a larger feature size. For laser direct write processes, typical substrate-to-target distances may be between about 10 microns and about 100 microns, and for pulsed laser deposition processes, typical substrate-to-target distances may be between about 3 centimeters and about 20 centimeters.

The target material 320, 320′ may include a target matrix 330, 330′ disposed on a target substrate 340, 340′. The target substrate 340, 340′ may include a variety of suitable materials. The specific target substrate 340, 340′ may be selected to provide sufficient mechanical support to the target matrix 330, 330′. Further, in the laser-direct write process (as shown in FIG. 3), the target substrate 340 may additionally be selected to allow at least partial transmission of selected laser pulse wavelengths. Suitable target substrates may include a variety of crystalline or amorphous ceramics, such as fused quartz, fused silica, or various glasses or polymers.

The target matrix 330, 330′ may be selected from a number of suitable matrix materials, including a variety of polymeric or organic binder materials. The specific matrix material may be selected based on a number of thermal, optical, chemical, and/or biologic properties. For example, the matrix 330, 330′ can include a binder material selected such that it will absorb energy from the laser pulse 310, 310′ such that it will be heated, evaporated, decomposed or otherwise chemically altered by a selected laser pulse 310, 310′ to release substances contained within the matrix 330, 330′. Further, the specific matrix material 330, 330′ may be selected such that the material will not damage or react with enzymes or chemicals to be deposited on substrate 120. Suitable matrix materials may include, for example, sodium dodecyl sulfate (SDS) and frozen aqueous solutions. Suitable matrix materials may also include a number of polymers including, for example, polybutyl methacrylate, polyvinylidene, and/or polylactic acid.

The laser pulse 310, 310′ may affect the matrix 330, 330′ in a number of ways. For example, some matrix materials 330, 330′ will be vaporized be the laser pulse 310, 310′. The vaporized matrix materials 330, 330′ will then release substances contained within the matrix materials 330, 330′. Further, in some embodiments, vaporization of the matrix materials 330, 330′ will propel the substances contained therein towards the substrate 120. Other matrix material, including some polymers, may be chemically altered or degraded by the laser pulse 310, 310′ to release substances contained therein. Further, some matrix materials may be frozen, and frozen matrix materials may be melted or evaporated to release substances contained therein.

The matrix material 330, 330′ may be provided in a number of suitable forms. For example, suitable matrix materials 330, 330′ may include a paste formed from polymers, binders, and a certain amount of solvent. Suitable solvents may include for example water, alcohols, buffers, 1-methyl-2-pyrrolidone, and/or any other suitable solvent. These pastes may be applied to the target substrate 340, 340′ and may have sufficient viscosity to adhere to the target substrate 340, 340′. In other embodiments, the paste may be heated or air dried to evaporate the solvent from the paste, thereby producing a dry or solid matrix 330, 330′ on the target substrate 340, 340′.

In other embodiments, the matrix material 330, 330′ can include a solid matrix. Suitable solids may be produced, for example, by freezing a liquid or paste matrix material onto a target substrate 340, 340′. In some embodiments, freezing may be desirable to protect various enzymes or other materials contained within the matrix 330, 330′ or to prevent evaporation of volatile solvents from the matrix material 330, 330′. In addition, the matrix material 330, 330′ may include a materials that is naturally solid over deposition temperature ranges. Such naturally solid materials may includes, for example, metal thin films which may be used as electrode materials.

One or more substances to be deposited on the substrate material 120 may be contained within the matrix material 330, 330′ such that when the matrix 330, 330′ is exposed to a suitable laser pulse 310, 310′, the substance will be deposited on the substrate material 120. These substances may include a variety of electrode materials, enzymes, and/or other chemicals.

A variety of suitable enzymes may be selected for deposition on the substrate 120. For example, in some embodiments, one or more enzymes may be selected to facilitate production of an electrochemical reaction. In one embodiment, enzymes having glucose as an enzymatic substrate may be selected. Such enzymes may react with glucose to produce a redox reaction or reaction products that can be subsequently detected by the meter 200, 210. Suitable enzymes may include, for example, glucose oxidase and glucose dehydrogenase.

In addition, the matrix material 330, 330′ may further include other chemicals such as redox mediators, buffers, or any other suitable chemical. These chemicals may be included in the same matrix 330, 330′ as selected enzymes such that these chemicals will be deposited at the same time as the selected enzymes. Alternatively, the chemicals may be deposited in a separate deposition step either before or after deposition of selected enzymes. These chemicals may include, but are not limited to, ruthenium hexamine chloride, potassium ferricyanide, potassium ferrocyanide, phosphate buffer, tris buffer, sucrose, glycerol, polyvinylalcohol, or triton. Any suitable, buffer, chemical mediator, detergent, or other chemical may be included.

The type of laser 300, 300′ may also be selected from a number of suitable laser types. The specific laser type and laser operating parameters can be chosen based on the material to be deposited, the target matrix 330, 330′ used, cost, efficiency, speed, and any other suitable parameter. Suitable lasers 300, 300′ may include, for example, carbon dioxide lasers, ruby red lasers, krypton-fluorine lasers, xenon-chlorine lasers, neodymium:YAG lasers, fluorine lasers, argon-fluorine lasers, nitrogen lasers, Excimer™ lasers, or any other suitable laser type. In some embodiments, the laser 300, 300′ may be selected to vaporize, melt, or chemically alter the target matrix 330, 330′. For example, in some embodiments, the laser 300, 300′ may be chosen to have a wavelength that will be at least partially absorbed by the target matrix 330, 330′.

In some embodiments, the laser 300, 300′ may be selected to prevent or minimize damage to the one or more materials to be deposited on the substrate 120. For example, in some embodiments, the one or more materials may include enzymes or other materials that may be inactivated and/or damaged by certain laser wavelengths. The laser 300, 300′ may be selected to minimize damage to or inactivation of the enzymes or chemicals to be deposited on the substrate 120. In addition, the laser 300, 300′ may be selected to prevent excess heating of the target matrix 330, 330′, as excess heating may damage enzymes or other materials to be deposited on the substrate 120.

In addition, the laser fluence or pulse characteristics may be selected based on a number of factors. For example, in some embodiments, the laser fluence or pulse duration may be selected to release substances contained within the target matrix 330, 330′ without damaging the materials to be deposited on the substrate 120. For example, in some embodiments, the laser pulse duration may be in the femtosecond, picosecond, or nanosecond range, and a fluence of about 0.05 to about 10 Joules/cm² will be selected to prevent inactivation of enzymes such as glucose oxidase or glucose dehydrogenase. In other embodiments, a higher fluence or longer pulse duration may be used. For example, higher fluences or longer pulses may be selected for deposition of some electrode materials such as gold or platinum.

In some embodiments, the substrate 120, target material 320, 320′, and/or laser 300, 300′ may be mobile with respect to one another. For example, in one embodiment, the laser 300, 300′ may be rastered across sections of the target material 320, 320′ to facilitate deposition of materials onto the substrate 120 in a desired pattern. Further, the laser 300, 300′, substrate 120, or a sample holder containing multiple substrates may be mobile, thereby allowing deposition on substrates for multiple test media 100. Alternatively or additionally, the target material 320, 320′ may be horizontally or rotatably mobile. Movement of the target 320, 320′ will allow regions of the target matrix 330, 330′ that have been previously exposed to the laser pulse 310, 310′, thereby removing some or all of the material to be deposited, to move out of the path of laser 300, 300′. In addition, the substrate 120, may be horizontally mobile, thereby allowing positioning of the substrate 120 during material deposition.

As noted, the laser pulse 310, 310′ can be configured to melt, vaporize, or chemically alter the matrix material 330, 330′, thereby releasing substances contained within the matrix 330, 330′ and depositing these substances on the substrate 120. In some embodiments, the laser 300, 300′ will vaporize or chemically alter solvents or polymers contained within the matrix material 330, 330′ and the vaporized or chemically altered material will be released as a stream 360, 360′ containing a vaporized or particulate material that will not be deposited on the substrate. The stream 360, 360′ may further be evacuated by a gas stream 370, 370′ configured to remove volatile or particulate matrix material. The gas stream 370, 370′ can include suitable inert gases, such as argon or nitrogen.

In one embodiment, multiple test media components may be produced or deposited using a laser deposition technique. For example, it may be desirable to produce one or more electrodes using a laser deposition technique and then to deposit one or more enzymes and/or other media components using a laser deposition technique. Use of the described laser deposition techniques for both production of electrodes and deposition of enzymes and other substances may facilitate manufacturing. For example, use of the same production process may increase throughput, allow the use of a single production chamber or similar production apparatuses, and allow similar control of component dimensions for each test media component.

Laser deposition processes, as illustrated in FIGS. 3-4 may be selected to deposit electrodes, enzymes, reaction mediators, buffers, and/or any other suitable substances directly onto the substrate 120. In these embodiments, the electrode shape and dimensions can be controlled by selecting appropriate laser characteristics, matrix type, target-to-substrate distance, and any other suitable factor. However, in some embodiments, it may be desirable to control a deposition pattern using a mask.

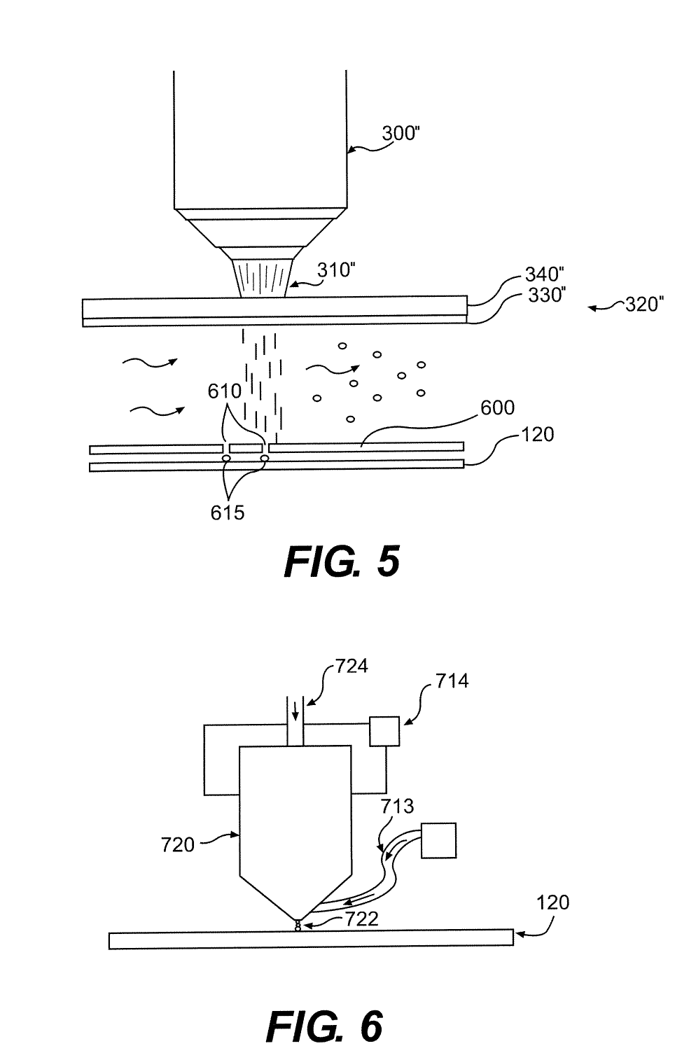

FIG. 5 illustrates a laser direct-write process, which includes a shadow mask 600. In this process, the mask 600 is first positioned in front of the substrate 120. Next, a laser 300″ will produce a laser pulse 310″, which is directed through a target material 320″. The laser pulse may be directed towards the target material 320″, thereby directing selected substances contained within a target matrix 330″ through the mask onto a suitable substrate 120. The mask 600, being positioned in front of the substrate 120, will include one or more openings 610 that define certain sections 615 of the substrate 120 on which the substances contained in the matrix may be deposited. Finally, the mask 600 can be removed to expose the deposited material on the substrate 120.

A variety of suitable masks 600 may be used. For example, in one embodiment, the mask 600 may include an adhesive shadow mask as described in U.S. Pat. No. 6,805,780 to Ryu, herein incorporated by reference in its entirety. Alternatively, any other mask that may be used to produce desired mesoscopic or microscopic component dimensions may be selected. For example, suitable masks may be produced using a photoresist.

If a mask 600 is used, other process parameters may be changed to speed production or otherwise improve the production process. For example, use of the mask 600 can allow wider laser pulses 310″ to be used. A wide pulse may allow deposition of a complete test media feature, such as an electrode, in a single pulse. This can increase production speeds and reduce laser operational costs.

FIG. 6 illustrates another method for producing a test media 100, according to an exemplary disclosed embodiment. This method includes deposition of aerosolized materials onto a substrate 120 using an aerosol-deposition system 700. The aerosol-deposition system 700 can include a deposition head 720 configured to focus the materials to specific sections of the substrate 120. Aerosol deposition processes can be used to produce test media components from a variety of different materials. Such components can include various electrode materials, enzymes, mediators, buffers, and/or any other substance needed to produce a suitable test media 100. Variations of aerosol deposition techniques for microelectronic fabrication are described in Renn et al., “Maskless deposition technology targets passive embedded components, ” Pan Pacific Symposium, (2002), herein incorporated by reference in its entirety.

The deposition system 700 may include a nebulizer, which may be configured to produce an atomized or aerosol stream 722. For example, as shown, the deposition head 720 is operably connected to an ultrasonic transducer 714. The ultrasonic transducer 714 will provide ultrasonic energy to the deposition head 720. The energized deposition head will thereby atomize fluid supplied through a fluid inlet 713.

The atomized material can be directed towards the substrate material 120 by deposition head 720. In one embodiment, deposition head 720 may be configured to produce a focused stream 722 of atomized material. The focused stream 722 may be directed towards the substrate using a gas stream supplied through an air inlet 724. In some embodiments, the deposition head 720 will be configured to produce a focused stream 722 having a certain size. For example, in some embodiments, it may be desirable to produce a stream 722 that will produce test media components, such as electrodes, reaction sites, etc, having a certain resolution.

It should be noted that a variety of suitable configurations may be used for deposition system 720. As noted, in the embodiment of FIG. 6, the deposition head 720 is operably connected to an ultrasonic transducer 714. In other embodiments, an atomized material may be produced using a deposition module fluidly connected with or contained within the deposition head. The atomized materials may then be focused onto the substrate 120 using an air supply 724. Further, although the nebulizer may include an ultrasonic transducer 714, any suitable nebulizer design may be selected.

In some embodiments, the focused stream 722 may be mobile with respect to substrate 120. The focused stream 722 can therefore be moved around the substrate to produce a component, such as an electrode, having a desired shape. Movement of the stream 722 relative to the substrate 120 can be accomplished by moving the deposition head 720, the substrate 120, or both.

A variety of substances may be deposited using the aerosol-deposition system 700. For example, as with laser deposition processes, any electrode material, enzyme, buffer, reaction mediator, and/or any other substance needed to produce a suitable test media may be deposited using the aerosol-deposition system 700. These substances may be provided in a number of suitable forms. For example, suitable materials may be provided as particulate suspensions, dilute pastes, or dilute biological solutions. Further, mixtures of several materials, such as enzymes, buffers, and/or reaction mediators may be used.

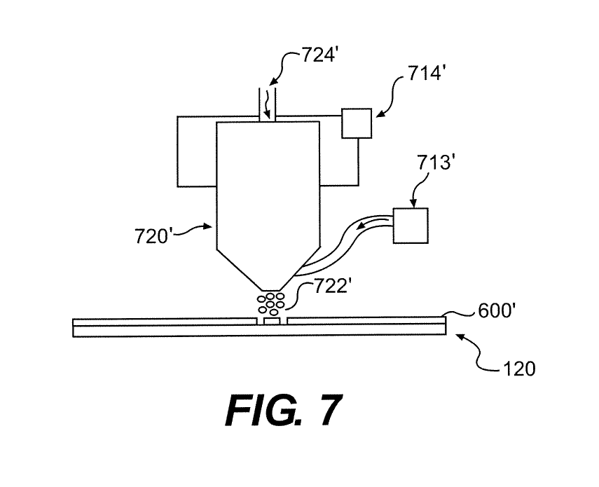

FIG. 7 illustrates another method for depositing substances on a substrate 120 using an aerosol-deposition system 700′. In this embodiment, an aerosol deposition system 700′ is again used to deposit one or materials on a substrate 120. However, as described previously for laser deposition processes, a mask 600′ is provided. Again, the mask 600′ may be configured to limit deposition to selected sections of the substrate 120, thereby producing desired feature shapes and/or dimensions.

If a mask 600′ is used, other process parameters may be changed to speed production or otherwise improve the production process. For example, in one embodiment, the aerosol-deposition system 700′ may be used with a mask 600′, and the focused stream 722′ may be made wide enough to cover the entire feature size and shape to be produced. In this way, the desired test media component may be produced using a single burst material.

As with laser deposition processes, aerosol deposition processes may be used to produce one or several components of a suitable test media 100. For example, in some embodiments, an aerosol deposition process may be used to produce one or more test media electrodes and to deposit enzymes or additional chemicals. Use of aerosol deposition for multiple test media components can reduce process complexity, increase throughput, and

With both laser deposition processes and aerosol deposition processes, the materials, such as electrode materials, may be further treated after deposition. For example, in some embodiments, in may be desirable to heat-treat or laser-sinter one or more components. Laser sintering may provide several advantages. For example, laser sintering can produce more sharply defined components, reduce edge effects, or facilitate production of certain material properties. In addition, lasers may be used to trim arrays, produce desired electrode shapes, or to test media components adjacent to electrodes.

### EXAMPLE 1

### Laser Direct-Write of Electrode Materials

Electrode materials were deposited onto Kapton™ films using a laser direct-write process. The Kapton film was cut to the shape of a glucose test strip. The conductive target matrix material was composed of either gold polymer paste or carbon graphite paste. The gold polymer paste was purchased from Gwent Electronic Materials, Ltd., (Product: C2041206D2). The carbon graphite paste was also purchased from Gwent Electronic Materials, Ltd., (Product: C2000802D2). The pastes were spread evenly as a wet layer onto a quartz ribbon. Transfer was completed using a Coherent Avia laser system.

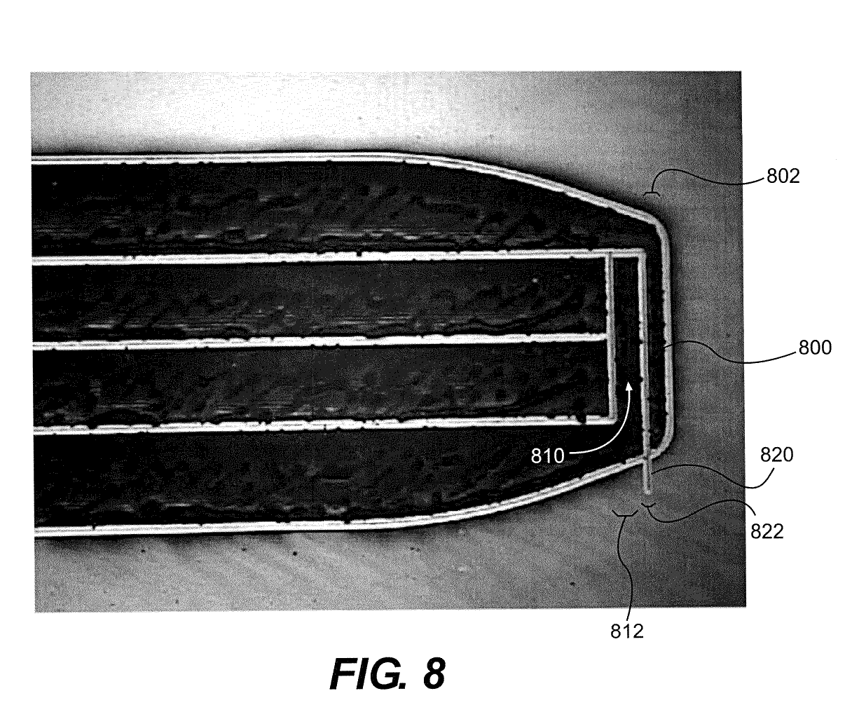

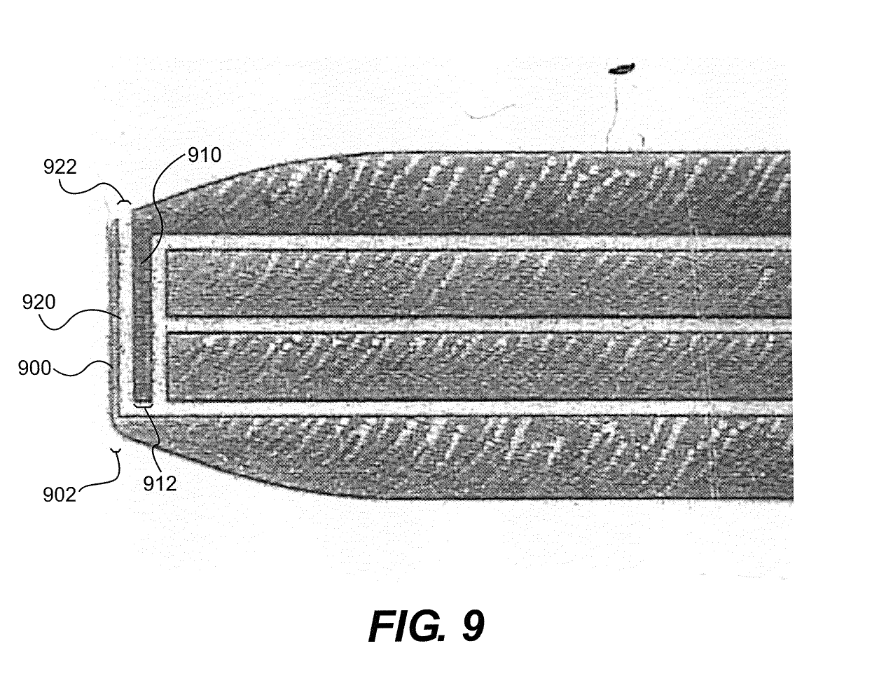

FIG. 8 provides a photograph of a sample test media with two electrodes 800, 810 produced using the carbon graphite paste. The electrodes included a cathode 800 and an anode 810. The cathode 800 had a width of approximately 241 microns, and the anode 810 had a width of approximately 381 microns. The gap 820 between that cathode 800 and anode 810 had a width 822 of approximately 125 microns. FIG. 9 provides a photograph of a sample test media with two electrodes 900, 910 produced using the gold paste. The electrodes included a cathode 900 and an anode 910. The cathode 900 had a width of approximately 150 microns, and the anode 910 had a width of approximately 280 microns. The gap 920 between that cathode 900 and anode 910 had a width 922 of approximately 200 microns.

Both electrode samples are shown as deposited. In some embodiments, the electrodes may be further shaped by heat treatment, laser sintering, or laser etching. Heat treatment, laser sintering, or laser etching may produce desired electrode structural properties and/or remove edge defects.

### EXAMPLE 2

### Aerosol Deposition of Chemistry Materials

Enzyme and mediators were spray deposited onto polyethylene terephthalate (PET) films using a SonoTek Micro-Mist™ dispensing system. Two different enzyme formulations were deposited. The first formulation included a glucose dehydrogenase solution comprising 100 mM phosphate buffer, 100 mM hexamine ruthenium chloride, 0.05% Triton X, 2% Celvol #203 polyvinyl alcohol, 5% sucrose, and 2500 units/ml glucose dehydrogenase. The second formulation included a glucose oxidase solution comprising 100 mM phosphate buffer, 100 mM hexamine ruthenium chloride, 0.05% Triton X, 2% Celvol #203 polyvinyl alcohol, 5% sucrose, and 2500 units/ml glucose oxidase. The formulations were deposited using a deposition head speed of 5 inches/second, a solution flow rate of 50 micrometers/minute, and an air pressure of 15 mm water. The flow rate was controlled using a syringe pump, and the nozzle was located approximately 0.5 inches away from the substrate surface.

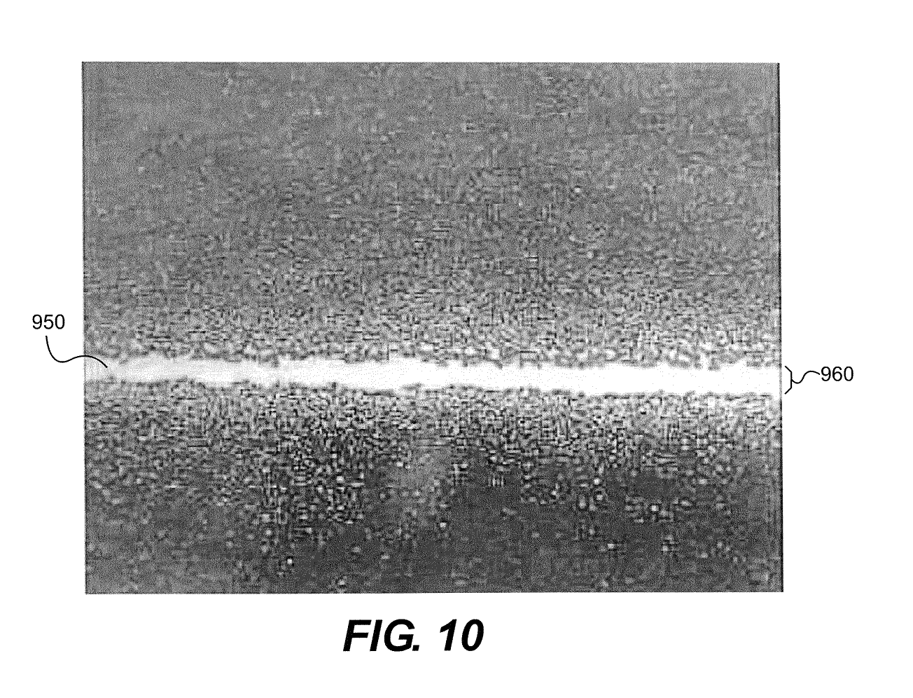

FIG. 10 provides a photograph of an enzyme formulation deposited using the methods of the present disclosure. A single enzyme line 950 is shown. The width 960 of the enzyme-mediator line 950 was approximately 600 microns. As noted previously, variations in process parameters may be selected to produce different sample dimensions or to deposit different enzyme-mediator formations.

As shown in FIG. 10, the enzyme-mediator formation was deposited as a line 950. However, the method of the present disclosure may be used to deposit suitable substances in a number of different configuration on a test media. For example, as noted previously, suitable test media 100 may include a reaction test site 150. The reaction test site can include a well, capillary, or other sample acquisition or retention means. In some embodiments, the aerosol deposition system or laser deposition systems of the present disclosure may be used to apply suitable mediators, buffers, enzymes, or other substances needed for a test reaction into a reaction site or section of a reaction site.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for manufacturing a test strip, comprising:
selecting a test strip substrate material;
positioning the test strip substrate material a predetermined distance from a matrix material disposed on a second substrate, wherein at least one enzyme to be deposited on the test strip substrate and having glucose as an enzymatic substrate is held within the matrix material; and
directing a laser pulse towards the matrix material to release at least a portion of the at least one enzyme having glucose as an enzymatic substrate from the matrix material and deposit the enzyme on the test strip substrate.

2. The method of claim 1, wherein the laser pulse passes through the second substrate before striking the matrix material.

3. The method of claim 2, wherein the second substrate includes at least one or fused quartz and fused silica.

4. The method of claim 1, wherein the enzyme includes glucose oxidase.

5. The method of claim 1, wherein the enzyme includes glucose dehydrogenase.

6. The method of claim 1, wherein the matrix further includes at least one of a buffer and a reaction mediator.

7. The method of claim 1, further including positioning a mask between the test strip substrate material and the second substrate material to define a region on the test strip substrate material where the enzyme having glucose as an enzymatic substrate is deposited.

8. The method of claim 1, wherein the matrix material includes a frozen material.

9. The method of claim 1, wherein the matrix material includes a polymer.

10. The method of claim 9 , wherein the polymer includes a polymer selected from the group including polybutyl methacrylate, polyvinylidene, and polylactic acid.

11. A method for manufacturing a test strip, comprising:
selecting a test strip substrate material;
positioning the test strip substrate material a predetermined distance from a matrix material disposed on a second substrate, wherein at least one electrode material to be deposited on the test strip substrate is held within the matrix material; and
directing a laser pulse towards the matrix material to release at least a portion of the at least one electrode material from the matrix material and deposit the electrode material on the test strip substrate.

12. The method of claim 11, wherein the laser pulse passes through the second substrate before striking the matrix material.

13. The method of claim 11, wherein the electrode material includes carbon.

14. The method of claim 11, wherein the electrode material includes at least one of gold and palladium.

15. The method of claim 11, further including positioning a mask between the test strip substrate material and the second substrate material to define a region on the test strip substrate material where the electrode material substrate is deposited.

16. A method for manufacturing a test strip, comprising:
selecting a test strip substrate material;
positioning the test strip substrate material a predetermined distance from a electrode matrix material disposed on a second substrate, wherein at least one electrode material to be deposited on the test strip substrate is held within the matrix material;
directing a laser pulse towards the electrode matrix material to release at least a portion of the at least one electrode material from the electrode matrix material and deposit the electrode material on the test strip substrate;
positioning the test strip substrate material a predetermined distance from an enzyme matrix material disposed on a third substrate, wherein at least one enzyme to be deposited on the test strip substrate and having glucose as an enzymatic substrate is held within the enzyme matrix material; and
directing a laser pulse towards the enzyme matrix material to release at least a portion of the is enzyme having glucose as an enzymatic substrate from the enzyme matrix material and deposit the enzyme on the test strip substrate.

17. The method of claim 16, wherein the enzyme includes glucose oxidase.

18. The method of claim 16, wherein the enzyme includes glucose dehydrogenase.

19. The method of claim 16, wherein the enzyme matrix material further includes at least one of a buffer and a reaction mediator.

20. The method of claim 16, wherein the electrode material includes carbon.

21. The method of claim 16, wherein the electrode material includes at least one of gold and palladium.

22. A method for manufacturing a test strip, comprising:
selecting a test strip substrate material; and
applying an aerosolized material including at least one enzyme having glucose as an enzymatic substrate to the test strip substrate.

23. The method of claim 22, wherein the enzyme includes glucose oxidase.

24. The method of claim 22, wherein the enzyme includes glucose dehydrogenase.

25. The method of claim 22, further including positioning a mask over the test strip substrate material to define a region of the test strip substrate on which the at least one enzyme having glucose as an enzymatic substrate will be applied.

26. A method for manufacturing a test strip, comprising:
selecting a test strip substrate material; and
applying an aerosolized material including at least one electrode material to the test strip substrate.

27. The method of claim 26, wherein the electrode material includes carbon.

28. The method of claim 26, wherein the electrode material includes at least one of gold and palladium.

29. The method of claim 26, further including positioning a mask over the test strip substrate material to define a region of the test strip substrate on which the enzyme will be applied.

30. A method for manufacturing a test strip, comprising:
selecting a test strip substrate material;
applying an aerosolized material including at least one electrode material to the test strip substrate; and
applying an aerosolized material including at least one enzyme having glucose as an enzymatic substrate to the test strip substrate.

31. The method of claim 30, wherein the enzyme includes glucose oxidase.

32. The method of claim 30, wherein the enzyme includes glucose dehydrogenase.

33. The method of claim 30, wherein the electrode material includes a carbon paste.

34. The method of claim 30, wherein the electrode material includes gold.
